(19) United States
(12) Patent Application Publication
(10) Pub. No.: US 2004/0014618 A1
(43) Pub. Date: Jan. 22, 2004

(54) Cyclic compound

(76) Inventor: Spencer Edwin Taylor, Camberley Surrey (GB)

Correspondence Address: NIXON & VANDERHYE, PC, 1100 N GLEBE ROAD, 8TH FLOOR, ARLINGTON, VA, 22201-4714, US

(21) Appl. No.: 10/149,985

(22) PCT Filed: Dec. 11, 2000
(86) PCT No.: PCT/GB00/04734

(30) Foreign Application Priority Data
Dec. 13, 1999 (GB) 9929433.2

(51) Int. Cl. C10M 129/14; C10L 1/18; C07C 69/94

(52) U.S. Cl. 508/479; 508/502; 508/510; 562/468; 562/476; 562/477

(57) ABSTRACT

A cyclic compound comprising m units of formula Ia and n units of formula (Ib) joined together to form a ring, wherein Y is a divalent bridging group which may be the same or different in each unit; R⁰ is H, a C₁-C₆ alkyl or a metal or ammonium cation; R⁵ is H or a C₁-C₆ alkyl; and j is 1 or 2; R³ is hydrogen, a hydrocarbyl or a hetero-substituted hydrocarbyl group; each of R¹, R² and R⁴, which may be the same or different, is hydroxyl, hydrogen, hydrocarbyl or hetero-substituted hydrocarbyl, with the proviso that at least one of R¹, R², R⁴ is hydroxyl, and m+n is 4 to 20, m is 1-8 and n is at least 3; and with the proviso that at least one of the conditions a) to e) below is satisfied: (a) the cyclic compound comprises at least one formula (Ia) unit in the form of a carboxylate anion, (b) the caclic compound comprises at least one formula (Ia) unit in the form of an alkaline earth metal or alkali metal carboxylate salt, (c) the cyclic compound comprises at least 2 units of formula (Ia) (d) the cyclic compound comprises an m+n value of 5 to 11, and (e) the cyclic compound is in the substantial absence of linear species comprising formula (Ia) and (Ib) units and/or the substantial absence of compounds of the formula (IIa) and (IIb) below: (IIa) (IIb).

The present invention relates to cyclic calixarene compounds containing within the calixarene ring at least one unit of salicylic acid or salt thereof. The compounds of the invention are particularly suitable as thermal stabilising additives in fuel and for use in lubricating oil compositions for medium or low speed diesel engines, especially four stroke engines. The invention also relates to fuel compositions and to lubricating oil compositions comprising said compounds, e.g. aviation fuel compositions.

In high speed aircraft, both civilian and military, the liquid fuel is combusted to produce power, but also is circulated in the aircraft as a heat exchange fluid to remove the excess heat generated at such speeds e.g. in lubricating oils. The fuel is thus maintained for long periods at high temperatures, which results in discoloration and decomposition to produce soluble coloured products and insoluble products such as gums, sediments and granular material; insoluble products can form deposits that reduce the heat exchange capacity and can block filters potentially causing loss of power. Soluble coloured by-products are unsightly and an indication of some decomposition. The cause of discoloration etc. may be from phenols, naphthenates and sulphur compounds and/or metals which are often present in the fuels.

In some oil fired devices, such as boilers and slow heating cookers, e.g. of the Aga type, kerosine oil fuel is passed down a narrow metal feed pipe to the combustion chamber where it is burnt. Parts of the pipe are sufficiently near the hot chamber for them to be heated to significant temperatures, resulting in the risk of thermal degradation of the fuel in the pipe, especially with slow feed rates and high residence times in the pipe. This degradation can form solid deposits which reduce the flow and ultimately stop it, causing the combustion to stop. To overcome this manufacturers of such devices have for many years recommended to their users that at least once each 6 months such pipe parts are cleaned of solid deposits of coke or other materials.

U.S. Pat. No. 5,478,367 describes the addition to diesel or jet fuel of a substituted unsaturated polyamine derivative dispersant to reduce particulate emissions on combustion and to reduce fouling i.e. deposition of insoluble deposits. The macrocyclic compounds preferably contain an N═C—N—C═O group and especially have fused rings, such as are made by reaction of a hydrocarbyl (e.g. fatty alkyl) succinic anhydride and a polyalkylene amine.

Canadian Patent Publ. 2067907 describes the addition to distillate jet fuels of hydroxylamines to stabilise them against degradation at elevated temperatures.

U.S. Pat. No. 5,468,262 describes addition to jet fuels of thermal stability additives which are prepared by reacting a polyamine, aldehyde and phenol to form a condensate which is then reacted with a succinic anhydride containing a polyolefin derived unsaturated group.

The additives are effective at 0.2% by weight.

EP-A-678568 describes addition to jet engine fuels of anti deposition agents which are derivatives of (thio)phosphonic acids.

Accordingly the present invention provides a cyclic compound comprising m units of the formula Ia.

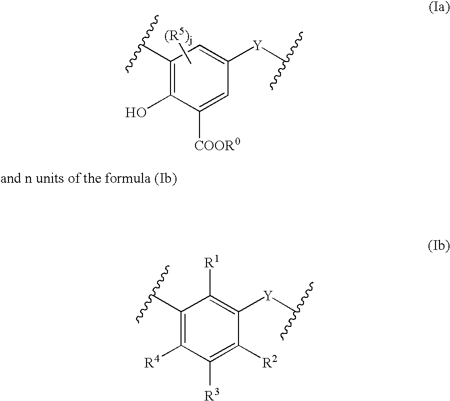

and n units of the formula (Ib)

joined together to form a ring,

wherein Y is a divalent bridging group which may be the same or different in each unit; R⁰ is H or (C₁-C₆) alkyl or is a metal or ammonium cation, (so the group C0₂R⁰ is a carboxylic salt); R⁵ is H or (C₁-C₆₀) alkyl; and j is 1 or 2;

R³ is hydrogen, a hydrocarbyl or a hetero-substituted hydrocarbyl group; each of R¹, R² and R⁴, which may be the same or different, is hydroxyl, hydrogen, hydrocarbyl or hetero-substituted hydrocarbyl, with the proviso that at least one of R¹, R², R⁴ is hydroxyl, and m+n is 4 to 20, m is 1-8 and n is at least 3 and preferably either R¹ is hydroxyl and R² and R⁴ are independently either hydrogen, hydrocarbyl or hetero-substituted hydrocarbyl, or R² and R⁴ are hydroxyl and R¹ is either hydrogen, hydrocarbyl or hetero-substituted hydrocarbyl; and m+n is from 4 to 20, m is from 1 to 8 and n is at least 3 and with the proviso that at least one of the following features (a) to (e) is met namely the cyclic compound comprises at least one of (a) at least one formula (Ia) unit in the form of a carboxylate anion especially at least 2 carboxylate anions (b) at least one formula (Ia) unit in the form of an alkaline earth metal or alkali metal carboxylate salt, preferably a potassium or sodium salt, especially a potassium salt (c) at least 2 e.g. 3 or 4 units of formula (Ia) (d) an m+n value of 5-11, preferably 7-9 and especially 8 and (e) the cyclic compound is in the substantial absence of linear species comprising formula (Ia) and (Ib) units and/or or the substantial absence of unreacted formula (Ia) and (Ib) units, (e.g. of formula Ia and IIb hereafter).

Preferably the cyclic compound has at least 2 of (a) (b) (c) (d) and (e) preferably 3, most preferably 4 and especially all 5 in particular (a) and (c), or (b) and (c), and especially (d). In a preferred embodiment the invention provides a cyclic compound wherein m is 2, n is 6, and both the units of formula (Ia) are in the form of a potassium carboxylate salt, and the cyclic compound comprises the substantial absence of linear species comprising formula (Ia) and (Ib) units and the substantial absence of unreacted formula (Ia) and (Ib) units. The cyclic compound is preferably a compound of the formula (I) with a ring structure.

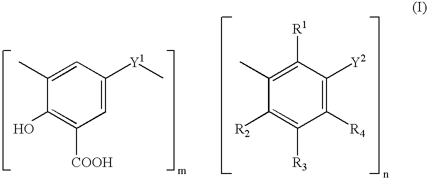

wherein Y¹ and Y² are divalent bridging groups, which may be the same or different; R³ is hydrogen, a hydrocarbyl or a hetero-substituted hydrocarbyl group; each of R¹, R² and R⁴, which may be the same or different, is hydroxyl hydrogen, hydrocarbyl or hetero-substituted hydrocarbyl, with the proviso that at least one of R¹, R², R⁴ is hydroxyl, and m+n is 4 to 20, m is 1-8 and n is at least 3 and wherein the compound of formula I comprises at least one of (a) at least one COOH grouping in the form of a carboxylate anion (b) at least one COOH grouping is in the form of an alkaline earth or alkali metal salt, preferably a potassium or sodium salt, especially a potassium salt (c) at least 2 e.g. 3 or 4 COOH groupings and (d) an m+n value of 5-11, preferably 7-9 and especially 8 and (e) the cyclic compound is in the substantial absence of linear species comprising salicylate or salicylic acid units and phenol units or the substantial absence of unreacted salicylate and/or salicylic acid units and phenol units.

Preferably the cyclic compound has at least 2 of (a) (b) (c) (d) and (e) preferably 3, most preferably 4 and especially all 5 in particular (a) and (c), or (b) and (c), especially (d). In a preferred embodiment the invention provides a cyclic compound wherein m is 2, n is 6, and both carboxyl groups are in the form of potassium carboxylate salt and the cyclic compound is in the substantial absence of linear species comprising salicylate or salicylic acid units and phenol units and the substantial absence of unreactived salicylate or salicylic acid units and phenol units.

When more than one salicylic acid unit is present in the ring (i.e. m>1), the salicylic acid and phenol units may be distributed randomly, although this does not exclude the possibility that in some rings there may be several salicylic acid units joined together in a row. Thus the m and n units may be joined in block and/or randomly. In the formula I Y¹ and Y² may each independently be a hydrocarbyl bridging group or be a hetero-substituted hydrocarbyl group or up to 50% mole of the totality of Y¹ and Y² group may be a hetero atom. The hydrocarbyl bridging group is preferably aliphatic and has a chain of 1-4 carbon atoms; preferably the group is of formula (CR⁷R⁸)_{d} e.g. (CHR⁸)_{d} where each of R⁷ and R⁸, which may be the same or different, represents hydrogen or hydrocarbyl e.g. of 1-6 carbons, such as methyl or ethyl and d is an integer of 1-4 preferably 2 or especially 1; advantageously the group is of formula (C⁶) or (CHR⁸)_{d} where R⁸ is as defined above preferably methyl or especially hydrogen. Y¹ and/or Y² may also represent a hetero-substituted hydrocarbyl group with a hetero atom, e.g. O, S or NH interrupting a chain of carbon atoms e.g. 2-4 carbon atoms, such as in CH₂OCH₂, CH₂SCH₂ or CH₂NHCH₂. Up to 50 mole % of the totality of Y¹ and Y² groups may be a hetero atom e.g. O or NH or especially 5, e.g. 1-50 mole % especially 8-20 mole % of said groups. Preferably Y¹ and Y² are hydrocarbyl groups, and the compound of formula I is sulphur free.

Each of R¹, R² and R⁴ represents hydroxyl, hydrogen, hydrocarbyl or hetero-substituted hydrocarbyl with the proviso that at least one of R¹, R² and R⁴ represents hydroxyl. Thus all three may represent hydroxyl as in a phloroglucinol derivative, or two as in a resorcinol derivative (i.e. the compound of formula I contains a resorcinarene group), or one as in a phenol derivative. Preferably either R¹ is hydroxyl and R² and R⁴ are independently either hydrogen (which is preferred), hydrocarbyl or hetero-substituted hydrocarbyl, or R² and R⁴ are hydroxyl and R¹ is either hydrogen, hydrocarbyl or hetero-substituted hydrocarbyl.

Regarding R¹ to R⁵ and R⁸, the term “hydrocarbyl” includes (C₁-C₆₀) alkyl such as t-butyl, t-amyl, s-butyl, isopropyl, octyl, nonyl, dodecyl and octadecyl. Alternatively the hydrocarbyl group may be derived from a polyolefin, for example polyethylene, polypropylene, polybutylene or a polyolefin copolymer, for example an ethylene/propylene copolymer, preferably derived from a polyisobutene. Alternatives include isoprene-butadiene, styrene-isoprene or styrene-butadiene block copolymers such as those disclosed in WO 96/40846, or ethylene-propylene and ethylene-butene-1 copolymers having molecular weights from 1500 to 2500 or 7500, as disclosed in U.S. Pat. No. 5,567,344 and U.S. Pat. No. 5,578,237. Mixtures of all the above may also be employed. Any hetero-substituted hydrocarbyl group has the heteroatom, preferably —O— or ═NH, interrupting a chain of carbon atoms, such as an alkoxy-alkyl group of 2-20 carbons. Each of R¹-R⁵ may otherwise be as described for R³ below.

The hydrocarbyl group for R¹, R² or R⁴ usually has 1-14 e.g. 1-6 carbons and is preferably saturated, especially an alkyl group e.g. methyl, ethyl, propyl, butyl or hexyl group. The hetero-substituted hydrocarbyl group has at least one e.g. 1-3 especially 1 hetero atom e.g. O, S or NH interrupting a chain of carbon atoms e.g. 2-20, or 2-6 carbons as in an alkoxy alkylene group such as ethoxy ethyl.

R³ is hydrogen, hydrocarbyl or a hetero-substituted hydrocarbyl group. Preferably R³ is hydrocarbyl or a hetero-substituted hydrocarbyl in at least R³ group in the compound of formula I, especially with n such groups in the molecule. The hydrocarbyl group may be alkyl, alkenyl, cycloalkyl, aryl, aralkyl and contains at least 1 especially at least 4 or at least 8 carbon atoms e.g. 4-40 carbons in particular with 8-20 carbons in a chain. Preferred are linear or branched alkyl e.g. of 8-24 or 8-20 carbons, such as decyl, dodecyl, tetradecyl, hexadecyl, octadecyl, lauryl, myristyl, stearyl, palmityl, propylene tetramer or alkenyl e.g. of 6-24 carbons such as oleyl, or cycloalkyl e.g. of 5-8 carbons such as cyclohexyl, aryl e.g. of 6-24 carbons such as phenyl, tolyl and alkylphenyl with 6-16 carbons in the alkyl e.g. dodecylphenyl and aralkyl e.g. of 7-26 carbons such as benzyl and alkyl substituted benzyl with 6-16 carbons in the alkyl e.g. dodecyl benzyl. R³ may also represent a polymeric hydrocarbyl group e.g. from a polyolefin group, especially from one or more olefins of 2-6 carbons such as ethylene, propylene, butene, isobutene; the polymeric groups may be from polyethylene, polypropylene, polybutene, an ethylene propylene copolymer or polyiso butene (which is preferred). Molecular weights of polymeric R³ groups may be 300-6000 e.g. 500-2000. In the compound of formula I, there may be different R³ groups in the same molecule.

In the compound of formula I, m is from 1 to 8 e.g. 1-4 especially 2 or in particular 1, while n is at least 3 e.g. 3-10, in particular 5-9 especially 6-8. The sum of m+n is 4-20, preferably 5-10 in particular 7-9, e.g. 6 or 8, or a mixture of compounds with m+n having the value of 6 and 8. Preferably m is 1 or 2 and m+n is 5-10 e.g. 8.

In preferred salixarenes Y, Y¹ or Y² is CH₂; R¹ is hydroxyl; R² and R⁴ are independently either hydrogen, hydrocarbyl or hetero-substituted hydrocarbyl; R³ is either hydrocarbyl or hetero-substituted hydrocarbyl; R⁰ is H or is substituted by an alkali metal ion, in particular a potassium ion; R⁵ is hydrogen or an alkyl group of 6 to 50 carbon atoms, preferably 4 to 40 carbon atoms, more preferably of 6 to 25 carbon atoms; j is 2 or preferably 1; and m+n has a value of at least 5, preferably at least 6, typically at least 8, where m is 1 or 2, preferably 1.

More preferably R² and R⁴ are hydrogen; R³ is hydrocarbyl, preferably alkyl of 10 greater than 4, preferably greater than 9 carbon atoms; R⁵ is hydrogen; and m+n is from 6 to 12 e.g. 8; m is 1 or 2.

In preferred compounds, R² and R⁴ are hydrogen, m is 1 or 2, n is 5, 6 or 7, m+n is 6 and/or 8, R¹ is hydroxyl, R³ is alkyl of 8-20 carbons e.g. dodecyl or octadecyl, or polyisobutenyl.

The metal in the salt form may be an alkali metal e.g. Li, Na, K, Rb or Cs, or an alkaline earth metal e.g. Mg or Ca, or ammonium or a quatenary ammonium salt e.g. of formula NR¹R²R³R⁴ wherein R¹-R⁴ are as defined above, especially with at least 3 and in particular 4 of them of less than 10 carbons.

For convenience the cyclic compounds are herein referred to as “salixarenes”.

For a review of calixarenes the reader is referred to ‘Monographs in Supramolecular Chemistry’ by C David Gutsche, Series Editor —J Fraser Stoddart, published by the Royal Society of Chemistry, 1989. Calixarenes having a substituent hydroxyl group or groups include homocalixarenes, oxacalixarenes, homooxacalixarenes and heterocalixarenes.

Salixarenes of the present invention may be made by reacting together appropriate amounts of the optionally substituted salicylic acid (or carboxylic ester), an optionally substituted phenol, and a carbonyl compound which is preferably an aldehyde e.g. formaldehyde, or acetaldehyde, in the presence of a base and optionally a catalyst. The reaction may be performed in the presence of sulphur if the compound of formula I is to contain combined sulphur.

The salixarenes may be made by a process comprising reacting together, in the presence of a basic catalyst, compounds of the formulas (IIa) and (IIb)

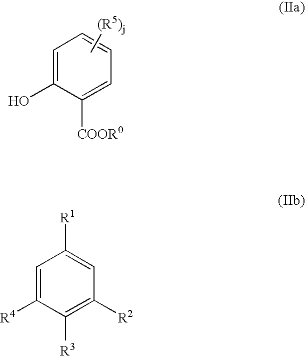

with an aldehyde of the formula O═CR⁷Rs e.g. O═CHR⁸, and optionally sulphur; where R⁰ to R⁵, R⁷, R⁸ and j are as defined previously.

Preferred basic catalysts are alkali metal hydroxides such as sodium hydroxide, potassium hydroxide and lithium hydroxide.

A proportion of the product may comprise linear molecules. Linear molecules are also composed of units having formulas (Ia) and (Ib) except that instead of the ends of the molecule being joined to form a ring, each end has a terminal group which is independently one of the following:

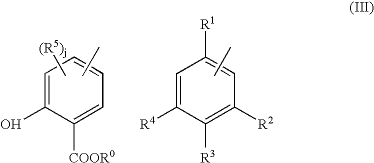

In the linear molecule the total number of units m+n is from 2 to 20, m is from 1 to 8 and n is at least 1. A further aspect of the invention provides the product of reacting compounds of the formulas (IIa) and (IIb) above with an aldehyde of the formula O═CHR⁶, and optionally sulphur; where R⁰ to R⁶ are as defined previously, which reaction product comprises at least 20% by weight of a cyclic compound comprising units of formulas (Ia) and (Ib) and no more than 80% of the linear version of said compound. Preferably the cyclic form comprises at least 40%, more preferably at least 60% and most preferably at least 80% by weight of the reaction product.

The process may be performed with an amount of base which is 0.01-0.2 equivalents per mole of the salicylic acid, with the base being effectively in catalytic amount.

The process may be performed in a melt or in the presence of water or a solution or suspension. The water may be added with the base e.g. aqueous sodium or potassium hydroxide solution, and/or a possible solvent for the formaldehyde (e.g. 10-30% formaldehyde solution), the amount of added water being usually 1-20% of the total S weight of the reactant e.g. 5-15%. Advantageously the process is performed with the water present initially, and then once reaction has started, then any water present as well as water produced as by product is removed e.g. by evaporation or distillation, whether under reduced pressure, and/or heating at above 110° C.

The process may also be performed in the presence of an inert organic liquid which may be a solvent for at least one of the reactants, e.g. the phenol and/or salicylic acid and/or a solvent or non solvent for at least one of the phenolic products, especially a salixarene e.g. of formula I and/or a linear phenolic species. The liquid is usually a hydrocarbon of atmospheric boiling point 100-160° C., such as toluene or a xylene or methylbenzene. The amount of liquid may be 10-90% of the total weight of solvent and reactants, especially 50-90%. Preferably the liquid is immiscible with water and can entrain water in its distillation and/or reflux, e.g. a Dean and Stark apparatus.

At the end of the reaction, the solvent if any may be evaporated to leave a crude product, or the reaction product (optionally with solvent) treated or used as such.

The direct product of the process to make the salixarenes may be used as such (i.e. without purification from other phenolic compounds) or may be converted from one salt form to another e.g. via ion exchange or by way of acidification to form the free acids, and then if desired formation of a salt by neutralisation. Preferably the direct product is purified to increase its content of cyclic and/or carboxylic species. The direct product may be distributed between a substantially water immiscible organic solvent e.g. an aliphatic liquid hydrocarbon, such as kerosene or hexane or an aromatic hydrocarbon such as toluene or xylene, and an aqueous medium. The medium may be basic e.g. with an alkali metal hydroxide or especially carbonate or bicarbonate, in which case the aqueous medium can extract the carboxylic species, while the non carboxylic species remains in the organic phase; the phases are then separated and then carboxylic salt recovered from the aqueous phase, or acidified to free carboxylic acid and recovered from an organic phase. The direct product may also be purified, when dissolved in organic solvent, by addition of a compound capable of producing a salt insoluble in the solvent e.g. addition of a barium salt (as such or in organic or aqueous solution) can cause precipitation of an insoluble barium carboxylate salt, which may be separated from the non carboxylic compounds. Acidification of the barium salt enables the free carboxylic acid to be recovered.

The direct product in organic solution is e.g. an aliphatic or aromatic hydrocarbon such as one described above, may be contacted with an anion exchange resin, so that the carboxylic compound(s) are bound to the resin, while the non carboxylic compounds are not. The treated resin may then be separated and treated with alkali metal hydroxide or alkoxide solution to regenerate the resin for reuse and to liberate the alkali metal salt of the carboxylic acid. The process may be batch wise or continuous (with a pair of resins in parallel, one for separation while the other is being regenerated). The resin is preferably a macroreticular one, and usually has quaternary ammonium cationic substituents; an example of such a resin is that sold under the Trade Mark Amberlyst 15.

The direct product may also be purified by shape selective separation by contact of the direct product (in acid or salt form) in organic solution with an inorganic porous solid, whose pores are insufficiently large to allow the salixarene to enter, but allow the linear products to enter. Preferred maximum diameter for pores are 100A, especially <70 or <50 A and especially above 20A. Examples of such porous solids are silica, alumina and silica/alumina as well as ceramic membranes, e.g. made of alumina or zirconia. The contact may be batch wise, e.g. passage of the direct product onto a bed of the solid and then elution of the cyclic compounds therefrom as the first eluted fraction; such beds may be in columns in parallel, one for separation of the cyclics while the other is being eluted of the other non cyclic materials. Preferably the separation is continuous e.g. in a cross flow made and/or with a tube of ceramic membrane, through the walls of which the non cyclics pass while the cyclics pass down the tube and are concentrated.

Preferably to make the compounds of formula I in which at least some, and preferably substantially all, the carboxylic groups are in salt form, the amount of base is at least 0.2 equivalents per mole of the salicylic acid, in particular 0.2-10, such as 0.9-5 especially 2-5 or about 1. The base chosen is usually the one which provides the cations required in the product, e.g. sodium or potassium hydroxide or carbonate to provide sodium or potassium salixarene salt. If desired the base for the reaction may be different, and then the reaction product salt may be converted into the desired salt e.g. via ion exchange. Thus an alkali metal base can be used in the reaction and the salt converted to for example quaternary ammonium or calcium, salt form. In addition instead of adding the salicylic acid as free acid into the reaction process a preformed salt thereof may be used, e.g. sodium or potassium salicylate, in the form for example of a solid or in aqueous solution.

Preferably to make the compounds with at least 2 carboxylic acid groups per ring, whether those groups are in acid or salt form or both, the process preferably involves reaction of the salicylic acid and the phenol in a molar percentage of at least 20% salicylic acid units based on the total of salicylic and phenol e.g. 20-90%, such as 20-40%. Advantageously the amount of base present is 0.2-8 equivalents, based on the equivalents of carboxyl groups in the salicylic acid e.g. 2-5 equivalents.

Preferably to make the salixarenes with a ring containing 5-11 aromatic rings (derived from the phenol and salicylic acid), especially with 7-9 such aromatic rings, and 1-3, in particular 2 or 3 salicylic rings, the process usually involves the presence of a base with a cation of appropriate size, the larger the cation the larger the ring. Thus while a spread of salixarene ring sizes is obtained, the average is lower for Li e.g. 5-7, than Na e.g. 6-8, than K 7-9 and Cs 9-11.

The fuel or lubricating composition e.g. jet fuel composition may also contain a non ring i.e. linear form of the compound of formula Ia/Ib I, i.e. with structural units as shown in the Formulae I but terminated usually by the phenol and/or salicylic acid units.

The present invention also provides the use of at least one of these “salixarenes” to reduce the discoloration on heating of fuel compositions e.g. jet fuel and fuel compositions comprising kerosine.

The preferred additive is dodecyl-salicylic calix[8]arene, which is a Salix[8]arene comprising 7 dodecyl substituted phenolic units and one salicylic acid unit joined by methylene bridges. Another preferred compound is the corresponding salixarene with 2 salicylic groups and 6 dodecylphenol units preferably at least partially as an alkali metal salt e.g. potassium salt and especially in the form of the dipotassium salt.

The additive may be present in the composition in amount of at least 1, at least Sppm, such as 1-1000, 5-1000 e.g. 5-500 especially 5-200 or 10-100 ppm based on the weight of the composition e.g. the jet fuel composition. The additive may be mixed with the jet or other fuel composition in the form of a concentrate in solution, e.g. in an aliphatic aromatic hydrocarbon in 20-80% w/w solution, or it may be added as such to give a solution in the fuel. More than one of the salixarenes may be present e.g. 2-4, especially differing only in the values of at least one of m and n, especially n.

The composition can comprise jet fuel. The composition can comprise kerosine, in particular in jet fuel. The main component of the jet fuel itself is usually a middle boiling distillate boiling point in the range 150-250° C. at atmospheric pressure and the fuel is usually kerosine which may be mixed with gasoline and optionally light petroleum distillate as in mixtures of gasoline and kerosene. The jet fuel may comprise mixtures of gasoline and light petroleum distillate, e.g. in weight amounts of 20-80:80-20 such as 50-75:50-25 which weight amounts may also be used for mixtures of gasoline and kerosene. The jet fuels for military use are designated JP4 to 8 e.g. JP4 as 65% gasoline/35% light petroleum distillate (according to US Mil. Spec. (MIL 5624G)), JP5, similar to JP4 but of higher flash point, JP7, a high flash point special kerosene for advanced supersonic aircraft and JP8, a kerosene similar to Jet A1 (according to MJL 83 133C). Jet fuel for civilian use is usually a kerosene type fuel and designated Jet A or Jet Al. The jet fuel may have a boiling point of 66-343° C. or 66-316° C. (150-650° F. e.g. 150-600° F.), initial boiling point of 149-221° C., e.g. 204° C. (300-430° F., e.g. 400° F.), a 50% boiling point of 221-316° C. (430-600° F.) and a 90% boiling point of 260-343° C. (500-650° F.) and API Gravity of 30-40. Jet fuel for turbojet use may boil at 93-260° C. (200-500° F.) (ASTM D1655-59T). Further details on aviation fuels may be obtained from “Handbook of Aviation Fuel Properties”, Coordinating Research Council Inc., CRC Report No. 530 (Society of Automotive Engineers Inc., Warrendale, Pa., USA, 1983) and on US military 25 fuels, from “Military Specification for Aviation Turbine Fuels”, MIL-T-5624P.

The jet fuel may be the straight run kerosene optionally with added gasoline, but preferably has been purified to reduce its content of components contributing to or encouraging formation of coloured products and/or precipitates. Among such components are aromatics and olefins and mercaptans. Thus the fuels may be purified to reduce their mercaptan content e.g. Merox fuels and copper sweetened fuels or to reduce their sulphur content e.g. hydrofined fuels or Merifined fuels. Merox fuels are made by oxidation of the mercaptans and have a low mercaptan S content (e.g. less than 0.005% wt S) such as 0.0001-0.005% but a higher disulphide S content (e.g. at most 0.4% or at most 0.3% wt S such as 0.05-0.25 e.g. 0.1-2%); their aromatic (e.g. phenolics) and olefins content are hardly changed. Hydrofined jet fuels are ones in which the original fuel has been hydrogenated to remove at least some of sulphur compounds e.g. thiols and under severe conditions to saturate the aromatics and olefins; hydrofined jet fuels have very low sulphur contents (e.g. less than 0.01% S by weight). Merifined fuels are fuels that have been extracted with an organic extractant to reduce or remove their contents of sulphur compounds and/or phenols. The jet fuel may also contain metals, either following contact with metal pipes or carried over from the crude oil; examples of such metals are copper, nickel, iron and chromium usually in amounts of less than 1 ppm e.g. each in 10-150 ppb amounts. Merox and hydrofined fuels are preferred and may be used in JP 4-8 jet fuels.

The fuel comprising kerosine may also be a fuel for combustion especially for non motive purposes, e.g. power generation, steam generation, and heating, especially for use 15 in buildings and for cooking, e.g. as described above. The fuel is particularly suitable for the devices e.g. boilers and slow cookers as described above in which there is localised preheating of the fuel before it is combusted. Such fuels are known as burning kerosine and may have the same physical properties as the kerosine based jet fuels described above, e.g. straight run kerosine, or kerosine modified to reduce its content of at least one of aromatics, olefins and sulphur compounds, as described above. The fuel may also contain metals as described above.

The fuel compositions of the invention contains the cyclic compound of formula Ia/Ib or I and may also contain at least one conventional additive e.g. for jet fuels or burning fuels such as an antioxidant, corrosion inhibitor, dispersant/detergent, (in particular in the case of hydroxy carboxylic acids (see below)), especially in amounts each of 1-1000 ppm, e.g. 20-200 ppm. The “salixarene” additives of formula I may be present in the composition especially with a dispersant; the dispersant is in particular one for solids known for use in fuels e.g. automotive burning or aviation fuels. Such dispersants usually have a polymeric carbon backbone with pendant groups containing nitrogen, which may be primary, secondary or tertiary, in cyclic or acyclic systems, and especially in amine, amide or imide groupings, in particular cyclic imide groups. The dispersants may also contain 1-5 polymer chains which are bridged by the nitrogen containing groups. Examples of such dispersants are the reaction products of polyisobutene succiic anhydride (PIBSA) and polyamines. Such dispersants are known compounds for dispersing particles of in non aqueous systems e.g. hydrocarbon systems. The weight ratio of “salixarene” to dispersant may be 99:1 to 10:90, especially 30:70 to 70:30. The additives and the fuel composition are preferably substantially ashless. Burning kerosine is usually substantially free of the above additives apart from that of formula I or Ia/Ib.

The fuel compositions of the invention containing the compounds of formula I, Ia, Ib, have an improved thermal stability as shown by a reduced tendency to discolour and/or produce solids on heating compared to the fuel alone (in the isothermal corrosion and oxidation test (ICOT based on ASTM D487 1)). In some cases the combination of the compounds of formula I and certain other hydroxy carboxylic acid derivatives imparts to some fuels further improved stability still, better than either additive alone. This synergistic behaviour is found with combinations of the compound of formula I, Ia, Ib and the hydroxycarboxylic acid in Merox fuels.

Thus in a preferred embodiment the invention also provides a blend comprising at least one compound of formula I, Ia, Ib and at least one hydroxy carboxylic acid (different from said compound) with at least one chain of at least 8 carbon atoms. The invention also provides a fuel composition comprising said blend and a fuel comprising kerosine and/or a jet fuel which is a Merox fuel, especially one which has a mean deposit forming tendency in the ICOT test according to ASTM D4871 of 80-120 mg deposit per litre of fuel, in particular 80-105 mg/l.

In the blend of this invention the weight ratio of the compound (c) of formula I, Ia, Ib to hydroxycarboxylic acid is usually 10-90:90-1, in particular 30-85:70-15 and especially 35-65:65-35. The amount of the blend in the fuel is usually 10-1000 ppm e.g. 30-200 ppm.

The hydroxycarboxylic acid contains in total at least I hydroxyl group e.g. 1-4 such as 2 or 3 but preferably 1 hydroxyl group. It usually contains a hydroxyl group on a carbon atom alpha, beta or gamma to the carbon atom to which the carboxylic acid group is bonded and may optionally have I or more hydroxyl groups elsewhere in the molecule: preferably the only hydroxyl group in the molecule is in the alpha, beta or gamma especially the beta position. The hydroxy acid may be of formula,

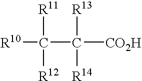

wherein one of R¹¹ and R¹³ represents hydrocarbyl and the other hydrogen or an organic group, each of R¹⁰, R¹² and R ¹⁴, which may be the same or different, represents hydrogen or an organic group, bonded via carbon or a heteroatom, which is O, N or S, with the proviso that at least one, and preferably only one of R¹⁰-R¹⁴ represents an organic group containing a carbon chain of at least 8 carbon atoms. Examples of the organic groups, bonded via carbon are alkyl, cycloalkyl, alkenyl, aralkyl or aryl, e.g. as described for R³ above, especially an alkyl group of 8-3000 carbons, in particular 8-24 carbons especially dodecyl, octadecyl, and 50-3000 carbons e.g. polyolefinyl such as from polyisobutene. Examples of the organic group bonded via nitrogen are amino groups with long chain hydrocarbyl group e.g. 8-24 carbons, or amido or imido groups from long chain carboxylic acids with 8-3000 carbons, e.g. 8-24 carbons such as fatty acids e.g. stearic and palmitic acids, or 50-3000 carbons e.g. polyolefinyl such as from a carboxylic derivative from polyisobutene such as PIBSA. In particular R¹ preferably represents hydroxyl/or hydrogen, R¹⁰ represents hydrogen or a long chain hydrocarbyl group of at least 8 carbons, especially 8-24 or 50-3000 carbons, R¹² represents hydrogen or alkyl of 1-6 carbons e.g. methyl or ethyl, R¹³ represents hydroxyl or hydrogen and R¹⁴ represents hydrogen or a amino, amido or imido group with a long chain aliphatic group or long chain mono or di acyl group, in particular a long chain succiic imide e.g. PIBSA. Especially R¹⁰ or R¹⁴ contains a long chain aliphatic group but not both. Preferred examples of the hydroxy carboxylic acid are N(long chain acyl) derivatives of beta hydroxy amino acids e.g. seine and threonine and long chain hydrocarbyl alpha hydroxy acids e.g. 1-hydroxy dodecanoic, 1-hydroxypalmitic and 1-hydroxystearic acids, 1-hydroxyl polyiso butenyl-1-carboxylic acid (from PIB aldehyde).

The invention also provides a fuel composition comprising liquid hydrocarbons, 30 at least a majority of which boil at atmospheric pressure at 251-350° C. (according to ASTM D86). The hydrocarbon fuel may be diesel oil (e.g. as defined in European Standard EN590: 1993, the disclosure of which is herein incorporated by reference), but is preferably gas oil (e.g. as defined in its European Standard the dislcosure of which is herein incorporated by reference), especially with a boiling range of 251-410° C. The gas oil is a hydrocarbon fraction boiling between kero sine and light lubricating oil. The gas oil may be straight run gas oil, from direct distillation of crude oil under atmospheric pressure, cracked gas oil, which has the above boiling range and is made by distillation of the product of cracking high boiling or other oils either thermally or catalytically, or vacuum gas oil, with the above boiling range made by vacuum distillation of a residue, e.g. from a crude oil distillation or cracked oil distillation, or visbroken gas oil, made by treating one of the above gas oils to reduce its viscosity. The liquid hydrocarbon in the fuel composition may also be a product of hydrotreating or hydrofining a gas oil as described above; more information on these is provided below.

The fuel composition can thus contain at least a majority of a gas oil e.g. 51-100% (of the liquid hydrocarbons) e.g. at least 70% such as 70-90%, with usually up to 30% by weight (of total liquid hydrocarbons) of liquid hydrocarbons boiling above or below the gas oil, e.g. kerosine bp 150-250° C. and heavy gas oils of Final Boiling Point 350-410° C. Blends comprising a majority of gas oil with a minority of kerosene, or a majority of gas oil with a minority in total of kerosine and the heavy gas oil are preferred. The fuel composition may contain one or more of the types of gas oil discussed above, preferably with straight run gas oil and at least one other gas oil, especially with at least 50% or at least 70% of the straight run gas oil and up to 30% in total of one or more other gas oils e.g. cracked gas oil.

The fuel of the invention may comprise diesel fuel itself e.g. for use in diesel engines for motive use such as automobiles, trucks and buses, rather than the cruder gas oil, which contains diesel and a wide range of liquid hydrocarbons of boiling point alone 25 and below diesel. The gas oil is preferred for non motive uses. The fuel may also comprise biofuel from vegetable and/or animal sources, such as rapeseed oil esters e.g. the methyl ester, especially in weight ratios to liquid hydrocarbons of bp 251-350° C. of 5-30:95-70.

The liquid hydrocarbons in the fuel of the invention may have at least one of and 30 preferably all of the following distillation properties 220-250° C. an initial bp (IBP) of 140-220° C., a 10% distillation point of 190-250° C., 50% distillation point of 220-250° C., 90% distillation point of 280-380° C. (e.g. 300-350° C.) 95% distillation point of 320-380° C. (e.g. 340-360° C.) and a Final Boiling Point of 290-385° C. (such as 330-360° C.).

The hydrocarbons may have Conradson Carbon contents (by weight) of 0.01-1 e.g. 0.01-0.1 or 0.1-1. They may have aniline points of 40-80° C. e.g. 60-75° C. (as in gas oils) or 10-40 e.g. 15-30 (as in diesel oil). They may have a Specific Gravity of 0.80-0.90, e.g. 0.82-0.88 or 0.82-0.86, and a minimum Flash Point of at least 38° C. (Closed, Abel.) or at least 55° C. such as 75-95° C. (Closed Pensky Martens). The cetane Number may be at least 40, 45, 49 or 51 such as 40-55, e.g. 45-48 or 49-54, but especially 40-49 or 45-49 as in gas oil. The S chemical analysis may be less than 0.5% sulphur compounds (expressed as elemental sulphur) such as 0.0001-0.5%, preferably less than 0.2% or 0.05% or 0.01%S, such as 0.05-0.2%S. The aromatic contents of the liquid hydrocarbons is usually less than 40% total aromatics e.g. 20-40% but especially less than 20% such as 5-15% total aromatics.

The liquid hydrocarbons e.g. gas oil or diesel preferably have been purified to reduce their content of components contributing to or encouraging formation of coloured products and/or precipitates or sulphur oxides on combustion. Among such components are aromatics and olefins and sulphur compounds. Thus the fuels may be purified to reduce their sulphur content e.g. hydrofined fuels or Merifined fuels. Hydrofined fuels are ones in which the original fuel has been hydrogenated to remove at least some of sulphur compounds e.g. thiols and under severe conditions to saturate the aromatics and olefins; hydrofined fuels have very low sulphur contents (e.g. less than 0.01% S by weight). Merifined fuels are fuels that have been extracted with an organic extractant to reduce or remove their contents of sulphur compounds and/or phenols. The fuel of the invention may also contain metals, either following contact with metal pipes or carried over from the crude oil; examples of such metals are copper, nickel, iron and chromium usually in amounts of less than 1 ppm e.g. each in 10-150 ppb amounts. Hydrofined fuels are preferred.

The fuel compositions of the invention include the cyclic compound of formula Ia/Ib or I and may also contain at least one conventional additive for automotive, heating or burner fuels e.g. an antioxidant, corrosion inhibitor, stabilisers, pour depressant, demulsiflers, antifoams, cetane improvers, lubricity additives, anti-static additives, dehazers, lubricity additives package compatibilisers and dispersant/detergent, (in particular in the case of hydroxy carboxylic acids (see below)), especially in amounts each of 1-1000 ppm, e.g. 20-200 ppm. The “salixarene” additives of formula I may be present in the composition especially with a dispersant; the dispersant is in particular one for solids known for use in fuels e.g. heating or burner fuels. Such dispersants usually have a polymeric carbon backbone with pendant groups containing nitrogen, which may be primary, secondary or tertiary, in cyclic or acyclic systems, and especially in amine, amide or imide groupings, in particular cyclic imide groups. The dispersants may also contain 1-5 polymer chains which are bridged by the nitrogen containing groups. Examples of such dispersants are the reaction products of polyisobutene succiic anhydride (PIBSA) and polyamines. Such dispersants are known compounds for dispersing particles of in non aqueous systems e.g. hydrocarbon systems. The weight ratio of “salixarene” to dispersant may be 99:1 to 10:90, especially 30:70 to 70:30. The additives and the fuel composition are preferably substantially ashless.

The fuel compositions of the invention containing the compounds of formula I, Ia, Ib, have an improved thermal stability as shown by a reduced tendency to discolour and/or produce solids on heating compared to the fuel alone. In some cases the combination of the compounds of formula I and certain other hydroxy carboxylic acid derivatives imparts to some fuels further improved stability still, better than either additive alone in particular with Merox fuels.

Thus the fuel composition may also include at least one hydroxy carboxylic acid 20 (different from said compound) with at least one chain of at least 8 carbon atoms.

The fuels of the invention may be used in combustion apparatus for motive use or in particular for non motive use in which the fuel is subjected to heating to a temperature e.g. of 100-400° C. such as 200-300° C. before combustion e.g. by proximity to the combustion chamber or otherwise (which may be at 500-700° C.). The feed pipes to the combustion chamber are usually made of metal e.g. copper or steel such as stainless steel, which may become corroded resulting in metal leaching into the oil encouraging degradation. The oil may be emitted into the chamber via a nozzle which becomes hot. The combustion may be in a vaporising burner in which metal in the burner chamber is heated by the flame and in turn vaporises the fuel; examples of such burners are vaporising and pot burners. The combustion is preferably in an atomising burner in which the fuel is atomised either directly, as in pressure jet burners (of low, medium or high pressure) (including simple and wide range burners) and blast burners, or indirectly as in rotary cup burners in which a sheet of fuel is made first and then atomised by contact with air. The atomising burners usually have a hot nozzle on and in which degradation deposits can form. The combustion apparatus may be used to produce heat directly as in industrial furnaces, e.g. for metals or ceramics, or industrial or domestic central heating or cooking as in slow cookers (e.g. of the Aga type) or for raising steam, e.g. process steam. Primarily the apparatus is used for raising power as in gas turbines for electrical power generation.

The invention also provides lubricating oil compositions suitable for ‘medium- or low-speed diesel engines, typically the four-stroke trunk-piston engine comprising said cyclic compound. Details of suitable lubricating oils and their other additives in addition to the cyclic compound are described in WO 9925677, the disclosure of which is hereby incorporated by reference.

The invention is illustrated in the following Examples.

### EXAMPLES

Preparation of 6 Dodecyl 2 Salicylic Calix(8)arene di Potassium Salt.

A reaction was performed between 234.5 g dodecylphenol (0.87 moles, 1 equiv)

17.25 g salicylic acid (0.125 moles, 0.152 equivs)

60 g paraformaldehyde (2.00 moles, 2.3 equivs) and

73.5 g 10 M potassium hydroxide (40% aqueous) (0.525 moles, 0.63 equiv)

A reaction apparatus was then set up incorporating the 5L flange flask, a flange lid and clip, overhead stirrer with paddle and PTFE stirrer gland, and double surface condenser and distillate collector. The reactor contents were heated by an electric mantle/thermocouple/Endotherm temperature controller system. The glassware from just above the mantle to just below the condenser was lagged with wool.

The reaction mixture was rapidly heated to 90° C., and the temperature then further increased very slowly at a rate of approximately 1° C. every 10 minutes. Water (77 ml) was collected over period of 7 hours, at the end of which the temperature had reached 140° C. The mixture was then allowed to cool overnight before being heated at about 140° C. for a further 2.5 hours.

The crude brown residue was dissolved in hexane and applied to an activated silica chromatography column (Kieselgel 60 Activated at 150° C. for 2 hr), from which it was separated by successive elution by hexane (35 ml), hexane/dichloromethane(DCM) (1: lvv) (80 ml), dichloromethane (50 ml), dichioromethane/tetrahydrofuran (THF) (50 ml) and tetrahydrofuran (50 ml). Quantitative recovery of 5 fractions 1-5 was obtained in the approximate weight ratio of 33, 23, 7, 15 and 21%. The fractions were analysed by IR, NMR (H and C Scanning electron microscopy and also tested for their thermal stabilising effect on a B99/l 11 aviation base fuel, which was POSF 2827 (from USAF).

The stability tests were Jet Fuel Thermal Oxidation Tests (JFTOT) and were performed as described in EP-A-660077 the disclosure of which is herein incorporated by reference. In the test the fuel contacts a heated standard aluminium tube under limited oxygen to produce deposits on the tube surface, the thickness of the deposits being determined either by colour (according to ASTM 3241) or ellipsometry according to EP-A-660077. The tubes were heated at 335° C. for 5 hr, and the deposit volume found from the thickness profile by ellipsometry.

The results of the thermal stability tests were as follows

| | | | Thermal deposition analysis | |
|---|---|---|---|---|
| | | | Concentration | Deposition |
| Fraction | Eluant | Mass % | (mgI⁻¹) | (ppb)* |
| 1 | Hexane | 33.4 | 8.3 | 21.5 |
| 2 | 1:1 | 22.5 | 5.9 | 148.5 |
| | Hexane/DCM | | 12.2 | 112.7 |
| 3 | DCM | 7.2 | 1.8 | 251.9 |
| 4 | 4:1 | 15.4 | 4.04 | 138.0 |
| | DCM/THF | | | |
| 5 | THF | 21.0 | 6.4 | 187.3 |

*The deposition results in the Table show the concentration of the various fractions in the fuel and the volume of deposit obtained expressed per volume of the fuel Fraction I eluted as a band with the solvent front suggesting low polarity material excluded from the silica pores.

The Hnmr spectrum of fraction 1 showed the presence of kerosene and hexane so the weight of solid in it is probably about 10% of the total crude residue.

The Hnmr and IA spectrum of Fraction 1 accorded with a cyclic salicylic acid/dodecyl phenol structure with rings bridged by methylene, while the SEM analysis and mass spectroscopy of the deposits on the JFTOT tubes shows the presence of K. Elemental analysis on Fraction 1 (after evaporation of kerosene and hexane accorded with 2CO₂ anionic groups and 2K cations per mole.

Fractions 2-5 are non carboxylic fractions of high or low molecular weight and of S poor activity, compared to Fraction 1.

1. A cyclic compound comprising m units of the formula Ia. 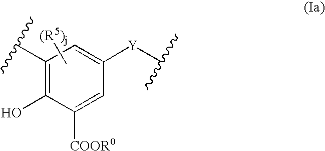
and n units of the formula (Ib) 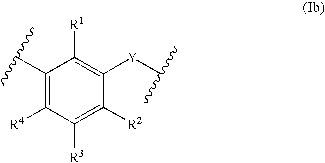
joined together to form a ring,
wherein Y is a divalent bridging group which may be the same or different in each unit;
R⁰ is H, a C₁-C₆ alkyl or a metal or ammonium cation;
R⁵ is H or a C₁-C₆₀ alkyl; and
j is 1 or 2;
R³ is hydrogen, a hydrocarbyl or a hetero-substituted hydrocarbyl group;
each of R¹, R² and R⁴, which may be the same or different, is hydroxyl, hydrogen, hydrocarbyl or hetero-substituted hydrocarbyl, with the proviso that at least one of R¹, R², R⁴ is hydroxyl, and
m+n is 4 to 20, m is 1-8 and n is at least 3; and
with the proviso that at least one of the conditions a) to e) below is satisfied:
(a) the cyclic compound comprises at least one formula (Ia) unit in the form of a carboxylate anion,
(b) the cyclic compound comprises at least one formula (Ia) unit in the form of an alkaline earth metal or alkali metal carboxylate salt,
(c) the cyclic compound comprises at least 2 units of formula (Ia)
(d) the cyclic compound comprises an m+n value of 5 to 11, and
(e) the cyclic compound is in the substantial absence of linear species comprising formula (Ia) and (Ib) units and/or or the substantial absence of compounds of the formula (IIa) and (IIb) below:

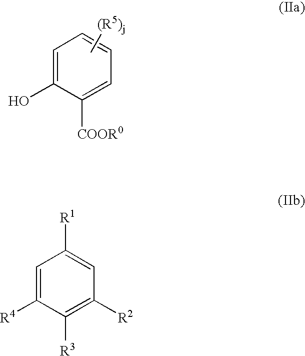

2. A compound as claimed in claim 1, wherein
either R¹ is hydroxyl and R² and R⁴ are independently either hydrogen, hydrocarbyl or hetero-substituted hydrocarbyl, or
R² and R⁴ are hydroxyl and R¹ is either hydrogen, hydrocarbyl or hetero-substituted hydrocarbyl.

3. A compound as claimed in claim 1 or 2, wherein Y is CH₂.

4. A compound as claimed in claim 2 or 3, wherein R¹ is hydroxyl, R² and R⁴ are hydrogen, and R³ is an alkyl of 8 to 20 carbon atoms.

5. A compound as claimed in any preceding claim, which comprises at least 2 of (a), (b), (c), (d) and (e).

6. A compound as claimed in claim 5, which comprises (c) and at least one of (a), (b), (d) and (e).

7. A compound as claimed in claim 5 or 6, which comprises at least three of (a), (b) (c), (d) and (e).

8. A compound as claimed in claim 7, which comprises (c), (d) and at least one of (a), (b) and (e).

9. A compound as claimed in any preceding claim, wherein
m is 2, and
n is 6.

10. A compound as claimed in any preceding claim, wherein at least one of COOR⁰ groups is in the form of an alkali metal salt, preferably, a lithium, sodium or potassium salt.

11. A compound as claimed in claim 10, wherein at least one of the COOR⁰ groups is in the form of a potassium salt.

12. A compound as claimed in any preceding claim, which is a 1-salix[8]arene comprising six dodecyl substituted phenolic units, and two salicylic acid unit joined by methylene bridges.

13. A compound as claimed in claim 12, which is in the form of its dipotassium salt.

14. A method of preparing a compound as claimed in any preceding claim, said method comprising
reacting together, in the presence of a basic catalyst, compounds of the formulae (IIa) and (IIb): 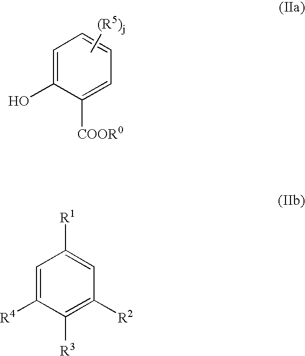
with an aldehyde of the formula O═CR⁷R⁸;
wherein j and R⁰ to R⁵ are as defined in claim 1,
wherein each of R⁷ and R⁸, which may be the same or different, represents hydrogen or a hydrocarbyl.

15. A method as claimed in claim 14, wherein the molar percentage of the compound of formula IIa is at least 20% of the total amount of compounds IIA and IIb.

16. A method as claimed in any one of claims 14 to 15, wherein the basic catalyst comprises alkali metal ions, preferably, potassium ions.

17. A method as claimed in any one of claims 14 to 16, which further comprises the step of recovering said cyclic compound in the substantial absence of linear species comprising formula (Ia) and (Ib) units and/or or the substantial absence of unreacted compounds of the formula (IIa) and (IIb).

18. A method as claimed in any one of claims 14 to 17, which comprises the step of converting the cyclic compound to the form of its alkali metal salt, preferably, its potassium salt.

19. A cyclic compound obtained by any one of claims 14 to 18.

20. A fuel or lubricating composition comprising a compound as claimed in any one of claims 1 to 13, or 19.

21. A composition as claimed in claim 20, wherein said compound is present in an amount of 1 to 1000 ppm based on the weight of the composition.

22. A composition as claimed in claim 20 or 21, wherein said composition comprises a jet fuel.

23. Use of a compound as claimed in any one of claims 1 to 13, or 19 as an additive for improving the thermal stability of a fuel or lubricating composition.

24. Use as claimed in claim 23, wherein said fuel or lubricating composition comprises a jet fuel.
